(19) United States
(12) Patent Application Publication
(10) Pub. No.: US 2005/0250185 A1
(43) Pub. Date: Nov. 10, 2005

(54) OGH fusion polypeptides and therapeutic uses thereof

(76) Inventors: Andrew J. Murphy, Croton-on-Hudson, NY (US); Lynn Macdonald, White Plains, NY (US); Mark W. Sleeman, Mahopac, NY (US)

Correspondence Address: REGENERON PHARMACEUTICALS, INC, 777 OLD SAW MILL RIVER ROAD, TARRYTOWN, NY, 10591, US

(21) Appl. No.: 11/008,727

(22) Filed: Dec. 9, 2004

Related U.S. Application Data
(60) Provisional application No. 60/529,036.
(60) Provisional application No. 60/548,115, filed on Feb. 27, 2004.

(51) Int. Cl. C07H 21/04; C07K 14/59; C07K 16/46

(52) U.S. Cl. 435/69.4; 435/320.1; 435/325; 435/326; 530/391.1; 530/399; 536/23.5; 530/398

(57) ABSTRACT

A fusion polypeptide comprising an orphan glyprotein hormone (OGH), OGH-family member, or a fragment thereof; a glycoprotein hormone subunit α1 or α2, or a fragment thereof, and optionally a peptide (P), and a fusion component (F). In one embodiment, the fusion protein comprises hOGH-hFc-hα2 or hTSH-hFc-hα1.

### CROSS-REFERENCE TO RELATED APPLICATIONS

This application claims the benefit under 35 USC § 119(e) of U.S. Provisional application 60/529,036 filed 12 Dec. 2003 and 60/548,415 filed 27 Feb. 2004, which applications are herein specifically incorporated by reference in their entireties.

### BACKGROUND OF THE INVENTION

1. Field of the Invention

The invention relates to orphan glycoprotein hormone (OGH) fusion proteins, methods of producing such fusion proteins, and methods for treating, diagnosing, or monitoring diseases or conditions using these fusion proteins.

2. Description of Related Art

Orphan glycoprotein hormone (OGH) was originally identified as a glycoprotein hormone beta subunit. Nakabayashi et al. (2002) J. Clin. Invest. 109:1445-1452 report the identification of alpha2 which combines with this new beta subunit (OGH/beta5) to form a heterodimeric hormone, termed “thyrostimulin”, which acts as an agonist of the thyroid stimulating hormone receptor (TSHR).

### BRIEF SUMMARY OF THE INVENTION

In a first aspect, the invention features a recombinant nucleic acid molecule encoding a thyroid-stimulating fusion polypeptide comprising R1, R2, and optionally a fusion component (F), and/or a peptide (P). wherein R1 is an orphan glycoprotein hormone (OGH), or an OGH family member, or a biologically active fragment thereof, and R2 is the human α1 or α2 or subunit or a biologically active fragment thereof which corresponds to R1, F is any component that enhances the functionality of the fusion polypeptide, and P is a peptide that allows R1 and R2 to assume a biologically active conformation.

In one embodiment, R1 is human OGH or fragment thereof capable of interacting with an α subunit to activate a receptor, for example, the thyroid stimulating hormone receptor (TSHR). In further embodiments, R1 is an OGH family member such as follicle stimulating hormone (FSH), thyroid stimulating hormone (TSH), leutinizing hormone (LH), or chorionic gonadotropin (CG).

R2 is a human α subunit capable of interacting with R1. When R1 is human OGH (hOGH, SEQ ID NO:7-8), R2 is human a2 (hα2, SEQ ID NO: 5-6) or fragment thereof capable of interacting with a β subunit to activate a receptor, for example, the thyroid stimulating hormone receptor (TSHR). When R1 is an OGH family member such as human FSH, human TSH (hTSH, SEQ ID NO:1-2), hLH, or hCG, R2 is human α1 (hα1, SEQ ID NO:1′-12), or fragment thereof capable of interacting with the R1 subunit to activate a receptor.

The optional fusion component (F) is any component that enhances the functionality of the fusion polypeptide. Thus, for example, a fusion component may enhance the biological activity of the fusion polypeptide, aid in its production and/or recovery, or enhance a pharmacological property or the pharmacokinetic profile of the fusion polypeptide by, for example, enhancing its serum half-life, tissue penetrability, lack of immunogenicity, or stability. In preferred embodiments, the fusion component is selected from the group consisting of a multimerizing component, a serum protein, or a molecule capable of binding a serum protein.

When the fusion component is a multimerizing component, it includes any natural or synthetic sequence capable of interacting with another multimerizing component to form a higher order structure, e.g., a dimer, a trimer, etc. In specific embodiments, the multimerizing component is selected from the group consisting of (i) an immunoglobulin-derived domain, (ii) a cleavable region (C-region), (ii) an amino acid sequence between 1 to about 500 amino acids in length, optionally comprising at least one cysteine residue, (iii) a leucine zipper, (iv) a helix loop motif, and (v) a coil-coil motif. In a more specific embodiment, the immunoglobulin-derived domain is selected from the group consisting of the Fc domain of IgG and the heavy chain of IgG. In a most specific embodiment the Fc domain of IgG is human FcΔ1 (a), an Fc molecule with a deletion of the region involved in forming the disulfide bond with the light chain. Human Fc (hFc) is shown in SEQ ID NO:13-14 and murine Fc (mFc) is shown in SEQ ID NO:3-4.

When the fusion component is a serum protein, the serum protein may be any serum protein or a fragment of a serum protein, such as alpha-1-microglobulin, AGP-1, albumin, vitamin D binding protein, hemopexin, afamin, or haptoglobin. When the fusion component is a molecule capable of binding a serum protein, it may be a small molecule, a nucleic acid, a peptide, or an oligosaccharide. It may also be a protein such as Fc gamma R1, ScFv, etc. In preferred embodiments, the fusion component is encoded by the nucleic acid, which encodes the fusion polypeptide of the invention. In some embodiments, however, such as when the fusion component is an oligosaccharide, the fusion component is attached post-translationally to the expressed fusion polypeptide.

In one embodiment, P is a peptide between 1-50 amino acids in length. In a more specific embodiment, P is a peptide between 30-40 amino acids in length. Still more specifically, P is between 30-40 amino acids in length, comprises at least one proline residue, and optionally, an O-linked oligosaccharide. In one specific embodiment, P comprises the C-terminal peptide (“CTP”) of human chorionic gonadotropin (HCG) having the nucleic acid and amino acid sequences of SEQ ID NO:9-10, or a variant or fragment thereof.

The recombinant nucleic acid molecule of the invention may further optionally comprise a signal sequence (SS) component. When a SS is part of the polypeptide, any SS known to the art may be used, including synthetic or natural sequences from any source, for example, from a secreted or membrane bound protein.

In one preferred embodiment, the recombinant nucleic acid molecule encodes a fusion polypeptide comprising R1-P-F, R1 is OGH or fragment thereof, P is CTP, and F is fusion component as the Fc domain of IgG1, IgG2, IgG3, IgG4, or any allotype within each isotype group. The components of the fusion polypeptide of the invention may be arranged in a variety of configurations, for example, R1-P-R2, F-R1-R2, R1-P-F-R2, R1-F-R2, SS-R1-F-R2, R1-R2-F, SS-R1-R2-F, R2-R1, etc., so long as the fusion polypeptide is capable of stimulating receptor activity. Examples of the fusion polypeptides of the invention include mFc-hOGH-CTP-hα2 (SEQ ID NO:15-16), hOGH-CTP-hFc-CTP-hα2 (SEQ ID NO:17-18), hTSH-mFC-hα1 (SEQ ID NO:19-20), hOGH-mFC-hα2 (SEQ ID NO:21-22), hOGH-CTP-hα2 (SEQ ID NO:23-24), and hOGH-hα2 (SEQ ID NO:25-26).

In a third aspect, the invention features a vector comprising a nucleic acid molecule of the invention. In further fourth and fifth aspects, the invention encompasses vectors comprising the nucleic acid molecules of the invention, including expression vectors comprising nucleic acid molecules operatively linked to an expression control sequence, and host-vector systems for the production of a fusion polypeptide which comprise the expression vector, in a suitable host cell; host-vector systems wherein the suitable host cell is, without limitation, a bacterial, yeast, insect, or mammalian cell. Examples of suitable cells include E. coli, B. subtilis, BHK, COS and CHO cells. Additionally encompassed are fusion polypeptides of the invention modified by acetylation or pegylation. Methods for acetylating or pegylating a protein are well known in the art.

In a related sixth aspect, the invention features a method of producing a fusion polypeptide of the invention, comprising culturing a host cell transfected with a vector comprising a nucleic acid molecule of the invention, under conditions suitable for expression of the protein from the host cell, and recovering the polypeptide so produced.

In a seventh aspect, the invention features a fusion polypeptide comprising R1, R2, and optionally a fusion component (F), wherein R1, R2, and FC are as defined above. In specific embodiments, the fusion polypeptide comprises the amino acid sequence of SEQ ID NO:16, 18, 20, 22, 24, or 26. The fusion polypeptide of the invention may activate a receptor as a monomer or as a multimer, e.g., for example, the fusion polypeptides may also form a dimer or higher order complex.

The fusion polypeptides of the invention are therapeutically useful for treating any disease or condition which is improved, ameliorated, or inhibited by administration of OGH, or an OGH family member, for example, in the treatment of infertility or in a thyroid-related conditions. Accordingly, in an eighth aspect, the invention features a therapeutic method for the treatment of an disease or condition, comprising administering a dimeric protein comprising a fusion polypeptide of the invention to a subject suffering there from. Although any mammal can be treated by the therapeutic methods of the invention, the subject is preferably a human patient suffering from or at risk of suffering from a condition or disease which can be improved, ameliorated, inhibited or treated with the fusion polypeptide of the invention.

In a ninth aspect, the invention features pharmaceutical compositions comprising an polypeptide of the invention with a pharmaceutically acceptable carrier. Such pharmaceutical compositions may comprise a monomeric or multimeric polypeptide, or nucleic acids encoding the fusion polypeptide. In a preferred embodiment, the pharmaceutical composition of the invention is a sustained release composition.

In a tenth aspect, the invention features kits containing a nucleic acid, or a monomeric or multimeric polypeptide of the invention, in a suitable container with instructions for use.

Other [LP1]objects and advantages will become apparent from a review of the ensuing detailed description.

### BRIEF DESCRIPTION OF THE FIGURES

FIG. 1: In vitro stimulation of TSHR (RLU-Cre-Luc unit×100) by OGH and TSH polypeptides (hTSH-mFc-hα1 ▪; hOGH-CTP-hα2▾; control ♦).

FIG. 2: Change in bodyweight (expressed as percentage of starting body weight) of C57BI6 mice injected via the tail vein with plasmid DNA encoding hOGH-CTP-hα2, hTSH-mFC-hα1 and sham control animals (hTSH-mFc-hα1 ◯; hOGH-CTP-hα2 empty diamond; control ▪).

FIG. 3: Change in bodyweight (expressed as percentage of body weight at the start of a high fat diet) of C57BI6 mice injected via the tail vein with plasmid DNA encoding hOGH-CTP-hα2, hTSH-mFC-hα1 and sham control animals (hTSH-mFc-hα1 ◯; hOGH-CTP-hα2 empty diamond; control ▪).

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

General Description

Orphan glycoprotein hormone (OGH) has been found to be associated with metabolic functioning, in particular, thyroid functioning. Transgenic animals over expressing OGH exhibit classic symptoms of hyperthyroidism, although their thyroids appear normal histologically. The present invention provides novel polypeptides, both monomers and multimers, capable of mimicking the biological activity of OGH and OGH family members. The fusion polypeptides of the invention are useful for treating metabolic diseases and disorders and symptoms of such diseases, for example, hypothyroidism, high serum triglycerides, etc. A preferred condition to be treated with the fusion polypeptides of the invention is obesity and/or obesity-related conditions. Further, the fusion polypeptides of the invention are useful when administered such that a low level of thyroid axis activation is achieved. The fusion polypeptides may also be used in treatment of infertility and related reproductive conditions.

Definitions

The term “nucleic acid molecule” as used herein refers to an oligonucleotide, nucleotide, and fragments or portions thereof, and to DNA or RNA of genomic or synthetic origin which can be single- or double-stranded, and represent the sense or antisense strand. Where “nucleic acid molecule” is used to refer to a specific polynucleotide sequence (a nucleic acid molecule encoding the human orphan glycoprotein hormone SEQ ID NO:7), “nucleic acid molecule” is meant to encompass polynucleotides that encode a polypeptide component that is functionally equivalent to the recited polypeptide, e.g., polynucleotides that are degenerate variants, or polynucleotides that encode biologically active variants or fragments of the recited polypeptide component. Similarly, “polypeptide” as used herein refers to an oligopeptide, peptide, or protein. Where “polypeptide” is recited herein to refer to an amino acid sequence of a naturally-occurring protein molecule, “polypeptide” and like terms are not meant to limit the amino acid sequence to the complete, native amino acid sequence associated with the recited protein molecule.

A “variant” of a human OGH. TSH, α1, α2, etc., polypeptide is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological or immunological activity can be found using computer programs well known in the art, for example, DNAStar software.

The term “derivative” as used herein refers to the chemical modification of a nucleic acid encoding a polypeptide component of the fusion polypeptide of the invention. Illustrative of such modifications would be replacement of hydrogen by an alkyl, acyl, or amino group. A nucleic acid derivative would encode a polypeptide which retains essential biological characteristics of the natural polypeptide.

By the term “active” or “therapeutic” agent, is meant a molecule capable of having a desired effect when delivered to the pre-selected target site, e.g., cell or tissue, including, but are not limited to, small molecules, hormones, growth factors, therapeutic biologics, antibodies and portions thereof, that are capable of having a desirable effect upon delivery to a target cell or tissue. In one example, the active agent is a compound capable of activating a target muscle receptor. The term “biologically active” refers to human OGH, TSH, α1, or α2 polypeptide having structural, regulatory, or biochemical functions of a naturally occurring polypeptides, e.g., ability to interact with each other to form a complex capable of activating a receptor such as TSHR.

The term “spacer” or “linker” means one or more molecules, e.g., nucleic acids or amino acids, or non-peptide moieties such as polyethylene glycol, which may be inserted between one or more component domains. For example, spacer sequences may be used to provide a restriction site between components for ease of manipulation. A spacer may also be provided to enhance expression of the polypeptide from a host cell, to decrease steric hindrance such that the component may assume its optimal tertiary or quaternary structure and/or interact appropriately with its target molecule. For spacers and methods of identifying desirable spacers, see, for example, George et al. (2003) Protein Engineering 15:871-879, herein specifically incorporated by reference.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; or (c) relieving the disease or condition, i.e., causing regression of the disease or condition. The population of subjects treated by the method of the invention includes subjects suffering from the undesirable condition or disease, as well as subjects at risk for development of the condition or disease.

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

As used herein, a “condition or disease” generally encompasses a condition of a mammalian host, particularly a human host, which is undesirable and/or injurious to the host. Thus, treating an endocrine disorder with an OGH fusion polypeptide which specifically binds cells expressing TSHR will encompass the treatment of a mammal, in particular, a human, who has symptoms reflective of decreased TSHR activation, or who is expected to have such decreased activation in response to a disease, condition or treatment regimen. Treating an endocrine-related condition or disease encompasses the treatment of a human subject wherein enhancing the activation of TSHR with the OGH polypeptide of the invention results in amelioration of an undesirable symptom resulting from the endocrine-related condition or disease.

Nucleic Acid Constructs and Expression

The present invention provides for the construction of nucleic acid molecules encoding OGH and OGH family member fusion polypeptides. The recombinant nucleic acid molecules of the invention may encode fragments of wild-type polypeptides, degenerative variants, or functionally equivalent variants thereof. Amino acid sequence variants of the components of the polypeptides of the invention may also be prepared by creating mutations in the encoding nucleic acid molecules. Such variants include, for example, deletions from, or insertions or substitutions of, amino acid residues within the amino acid sequence of the polypeptides. Any combination of deletion, insertion, and substitution may be made to arrive at a final construct, provided that the final construct possesses the ability to mimic the desired biological activity including, but not limited to, stimulating thyroid activity.

These nucleic acid molecules are inserted into a vector that is able to express the fusion polypeptides of the invention when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the polypeptides of the invention under control of transcriptional and/or translational control signals.

Expression of the nucleic acid molecules of the invention may be regulated by a second nucleic acid sequence so that the molecule is expressed in a host transformed with the recombinant DNA molecule. For example, expression may be controlled by any promoter/enhancer element known in the art. Promoters which may be used to control expression of the polypeptide molecules include, but are not limited to, a long terminal repeat (Squinto et al. (1991) Cell 65:1-20); SV40 early promoter region, CMV, M-MuLV, thymidine kinase promoter, the regulatory sequences of the metallothionine gene; prokaryotic expression vectors such as the b-lactamase promoter, or the tac promoter (see also Scientific American (1980) 242:74-94); promoter elements from yeast or other fungi such as Gal 4 promoter, ADH, PGK, alkaline phosphatase, and tissue-specific transcriptional control regions derived from genes such as elastase I.

Expression vectors capable of being replicated in a bacterial or eukaryotic host comprising the nucleic acid molecules of the invention are used to transfect the host and thereby direct expression of such nucleic acids to produce the fusion polypeptides of the invention. Transfected cells may transiently or, preferably, constitutively and permanently express the polypeptides of the invention. When the polypeptide so expressed comprises a fusion partner component which is a multimerizing component capable of associating with a multimerizing component of a second polypeptide, the monomers thus expressed then multimerize due to the interactions between the multimerizing components to form a multimeric polypeptide (see, for example, WO 00/18932, herein specifically incorporated by reference).

The fusion polypeptides of the invention may be purified by any technique known in the art. When the polypeptides of the invention comprise a multimerizing component which spontaneously forms a multimer with another polypeptide, the purification techniques used allow for the subsequent formation of a stable, biologically active multimeric polypeptide. For example, and not by way of limitation, the polypeptides may be recovered from cells either as soluble proteins or as inclusion bodies, from which they may be extracted quantitatively by 8M guanidinium hydrochloride and dialysis (see, for example, U.S. Pat. No. 5,663,304). In order to further purify the polypeptides, conventional ion exchange chromatography, hydrophobic interaction chromatography, reverse phase chromatography or gel filtration may be used.

R1 and R2 Polypeptide Components

In one embodiment, the fusion polypeptides of the invention comprise R1 which is selected from the group consisting of human OGH, a variant of hOGH, or a fragment of hOGH which is able to interact with R2 to form a complex capable of activating the TSH receptor. It will be readily apparent to one of skill in the art that numerous variants of OGH can be obtained which will retain substantially the same functional characteristics as the wild-type hOGH. The term “OGH or a fragment thereof” is intended to encompass not only the complete wild-type subunit, but also insertional, deletional, and/or substitutional variants thereof. Human OGH, including its native signal sequence, is shown in SEQ ID NO:7.

The fusion polypeptides of the invention comprise human α2 or α1 glycoprotein hormone subunit, or a variant or fragment thereof, designated α2, that is able to interact with R1 to form a complex capable of activating the desired receptor. The term “α2, α2 variant, or fragment thereof” is intended to encompass not only the complete wild-type subunit, but also insertional, deletional, and/or substitutional variants thereof. The mature form of human α2 (without signal sequence) is shown in SEQ ID NO:6 and hα1 in SEQ ID NO:12.

The fusion polypeptides of the invention are capable of enhancing the biological activity of OGH, as measured, for example, with a bioassay, or ELISA for free and/or bound ligand. Bioassays may include cAMP assays in which cAMP release is measured following stimulation of cells expressing TSHR with the fusion polypeptides of the invention.

Those of skill in the art will readily appreciate that changes can be made to any of the above amino acid sequences without substantially affecting the function of the polypeptide in carrying out a subject method. The amino acid sequence of the fusion polypeptide of the invention may be altered in various ways known in the art to generate targeted changes in sequence. Amino acid substitutions of interest include those that result in a reduced immune response in the individual being treated with the fusion polypeptide of the invention, and/or result in enhanced pharmacokinetic properties relative to wild-type component sequences. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; addition of a fatty acid or lipid; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the instant invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability, e.g., a polyethylene glycol (PEG) moiety may be attached, directly or via a linker, to one or more amino acids in the soluble fusion polypeptide.

Fusion Components

The fusion component (F) is any component that enhances the functionality of the fusion polypeptide, including for example, enhancing biological activity, production, recovery, or a pharmacological or pharmacokinetic property, e.g., serum half-life, tissue penetrability, lack of immunogenicity, or stability. In preferred embodiments, the F is selected from the group consisting of a multimerizing component, fusion partner, a targeting protein, a serum protein, or a molecule capable of binding a serum protein.

By the term “targeting protein” is meant a molecule, e.g., a protein or fragment thereof that specifically binds with high affinity a pre-selected cell surface protein, such as a receptor that is present to a greater degree at a pre-selected target site than on any other body tissue. Examples of targeting proteins include, but are not limited to, antibodies and portions thereof that bind a pre-selected cells surface protein with high affinity. By “high affinity” is meant an equilibrium dissociation constant of at least 10⁻⁷ molar, as determined by assay methods known in the art, for example, BiaCore analysis.

By the term “multimerizing component” is meant a component which allows a single polypeptide to form a multimer with one or more other polypeptides. Preferably, the multimeric polypeptide is a dimer. In some embodiments, the multimerizing component comprises an immunoglobulin-derived domain from, for example, human IgG, IgM or IgA, or comparable immunoglobulin domains from other animals, including, but not limited to, mice. In specific embodiments, the immunoglobulin-derived domain may be selected from the group consisting of the constant region of IgG, the Fc domain of IgG, an Fc-protein and the heavy chain of IgG. The Fc domain of IgG may be selected from the isotypes IgG1, IgG2, IgG3, and IgG4, as well as any allotype within each isotype group. In addition, the multimerizing component may be an Fc fragment or an amino acid sequence of 1 to about 200 amino acids in length containing at least one cysteine residue. In other embodiments, the multimerizing component is cysteine, or a short, cysteine-containing peptide. In addition, the multimerizing component may be unrelated to immunoglobulins and be, for example, a leucine zipper, a helix loop motif, or a coiled-coil motif.

Optional Component Spacers

The components of the fusion polypeptides of the invention may be connected directly to each other or be connected via spacers. Generally, the term “spacer” (or linker) means one or more molecules, e.g., nucleic acids or amino acids, or non-peptide moieties, such as polyethylene glycol, which may be inserted between one or more components. For example, spacer sequences may be used to provide a desirable site of interest between components for ease of manipulation. A spacer may also be provided to enhance expression of the polypeptide from a host cell, to decrease steric hindrance such that the component may assume its optimal tertiary structure and/or interact appropriately with its target molecule. For spacers and methods of identifying desirable spacers, see, for example, George et al. (2003) Protein Engineering 15:871-879, herein specifically incorporated by reference. A spacer sequence may include one or more amino acids naturally connected to a receptor component, or may be an added sequence used to enhance expression of the fusion protein, provide specifically desired sites of interest, allow component domains to form optimal tertiary structures and/or to enhance the interaction of a component with its target molecule. In one embodiment, the spacer comprises one or more peptide sequences between one or more components which is (are) between 1-100 amino acids, preferably 1-25. In one specific embodiment, the spacer is a three amino acid sequence; more specifically, the three amino acid sequence of Gly Pro Gly. A spacer having 10 or 25 amino acids may be informally indicated as “linker10” or “linker25”.

Therapeutic Uses

The fusion polypeptides of the invention are therapeutically useful for treating any disease or condition which is improved, ameliorated, inhibited or prevented by administration of OGH. A non-exhaustive list of specific conditions improved by administration of OGH include clinical conditions that are characterized by hypothyroidism; metabolic disorders such a elevated serum triglycerides, serum glucose, and/or serum cholesterol; modulation of insulin levels; and weight gain such as that associated with hypothyroidism, metabolic disorders, or other causes of weight gain.

Suitable Subject for Treatment

A suitable subject for treatment is a human diagnosed as suffering from hypothyroidism, a metabolic disorder characterized by elevated serum triglycerides, elevated serum glucose, and/or elevated serum cholesterol. A suitable subject for treatment also includes a human suffering from obesity.

Combination Therapies

In numerous embodiments, the fusion polypeptides of the invention may be administered in combination with one or more additional compounds or therapies. For example, multiple fusion polypeptides can be co-administered, or one or more polypeptides can be administered in conjunction with one or more therapeutic compounds. A benefit of the combined use of the fusion polypeptide of the invention with a second therapeutic agent is that it provides improved efficacy and/or reduced toxicity of either therapeutic agent.

Methods of Administration

The invention provides methods of treatment comprising administering to a subject an effective amount of a fusion polypeptide of the invention. In a preferred aspect, the fusion polypeptide is substantially purified (e.g., substantially free from substances that limit its effect or produce undesired side-effects). The subject is preferably a mammal, and most preferably a human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, intraocular, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents. Pulmonary administration can also be employed, e.g., by use of an inhaler or nebulizer, and formulation with an aerosolizing agent.

In another embodiment, the active agent can be delivered in a vesicle, in particular a liposome, in a controlled release system, or in a pump. In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), by direct injection, or by use of microparticle bombardment, or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

A composition useful in practicing the methods of the invention may be a liquid comprising an agent of the invention in solution, in suspension, or both. The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment.

Pharmaceutical Compositions

The present invention also provides pharmaceutical compositions comprising an OGH-α2 fusion polypeptide of the invention. Such compositions comprise a therapeutically effective amount of one or more fusion polypeptide(s), and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The fusion polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment of the invention, an fusion polypeptides of the invention are formulated in a sustained-release formulation. Sustained release formulations for delivery of biologically active peptides are known to the art. For example, U.S. Pat. No. 6,740,634, herein specifically incorporated by reference in its entirety, describes a sustained-release formulation containing a hydroxynaphtoic acid salt of a biologically active substance and a biodegradable polymer. U.S. Pat. No. 6,699,500, herein specifically incorporated by reference in its entirety, discloses a sustained-release formulation capable of releasing a physiologically active substance over a period of at least 5 months.

The amount of the fusion polypeptide that will be effective for its intended therapeutic use can be determined by standard clinical techniques based on the present description. In addition, in vitro assays may optionally be employed to help identify optimal dosage ranges. Generally, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. The amount of compound administered will, of course, be dependent on the subject being treated, on the subject's weight, the severity of the affliction, the manner of administration, and the judgment of the prescribing physician.

Cellular Transfection and Gene Therapy

The present invention encompasses the use of nucleic acids encoding the fusion polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.”

In another aspect, the invention provides a method of increasing OGH levels in a human or other animal comprising transfecting a cell with a nucleic acid encoding a polypeptide of the invention, wherein the nucleic acid comprises an inducible promoter operably linked to the nucleic acid encoding the polypeptide. For gene therapy procedures in the treatment or prevention of human disease, see for example, Van Brunt (1998) Biotechnology 6:1149-1154.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Cre-Luc Activation of mTSHR by OGH Tethered Constructs

cDNA encoding either human OGH fused to CTP polypeptide, human a2 glycoprotein hormone, and/or mouse or human Fc fused in any order were constructed using standard molecular biology techniques. The cDNAs were subcloned into a plasmid such that expression of the proteins is driven by an ubiquitin promoter. Constructs included hOGH-mFc-hα2, hOGH-CTP-mFc-hα2, hOGH-linker10-mFc-hα2, hOGH-linker25-mFc-hα2, hOGH-CTP-hα2, hOGH-hFc-hα2, hOGH-CTP-hFc-hα2, hOGH-CTP-hFc-CTP-hα2, hOGH-linker25-hFc-hα2, and hOGH-hα2.

The in vitro activity of the single chain polypeptides was determined by activation of the mouse thyroid stimulating hormone receptor (mTSHR). Plasmids expressing each of these constructs was transiently transfected into CHO cells and after 48 hours, supernatants were collected. cDNA encoding mTSHR were subcloned into a plasmid such that expression of the receptor is driven by a CMV promoter. This construct were transfected into FSC11 cells (HEK293 cells that are stably transfected with a Cre-Luc plasmid) to make a stable cell line expressing both the mTSHR and Cre-Luc reporter plasmid. Cre-Luc is a cAMP response element fused to a luciferase gene. When cAMP is released, it binds to Cre causing luciferase expression, which can be quantitated. 75 ul, 50 ul or 25 ul of the supernatants (supe) from the OGH single chain glycoproteins were incubated with FSC11 cells that stably express the mTSHR and Cre-Luc reporter plasmid for 6 hours. After 6 hours, cells were assayed for activation of receptor using a standard luciferase assay (Bright-Glo, Promega).

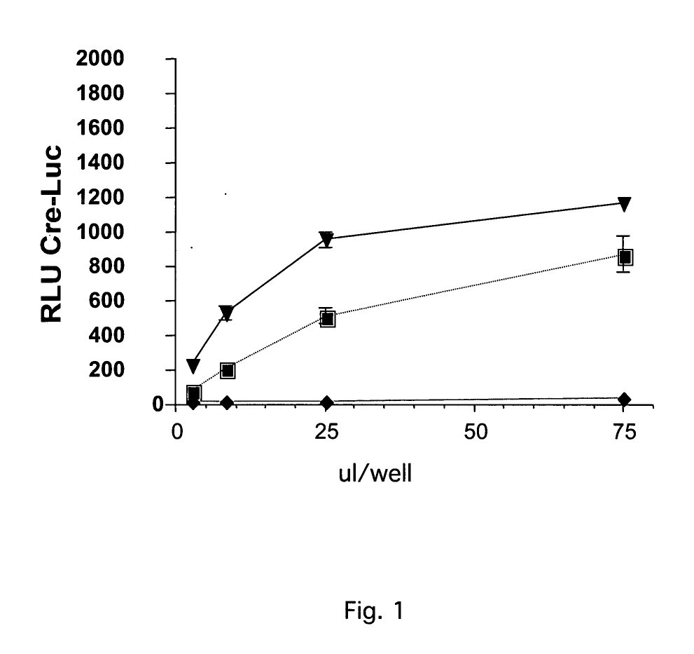

Results. When a stretch of amino acids coding for mFC, hFC and/or CTP was fused between the hOGH and hα2 components or TSH and hα1 components, the resulting single chain polypeptide was able to activate the mouse TSHR or hTSHR (FIG. 1; control: empty vector).

### Example 2

### OGH/TSH-Fc DNA Tail Vein Injection

Normal C57 mice (wild-type) were injected via the tail vein with plasmid DNA designed to express a constitutive low level of the OGH-Fc or TSH-Fc single chain fusion polypeptides or control (hTSH-mFC-hα1 (SEQ ID NO:20), hOGH-CTP-hα2 (SEQ ID NO:24), hOGH-mFC-hα2 (SEQ ID NO:22), mFc only (SEQ ID NO:14), or sham (control). Expression of the single chain fusion polypeptides was driven by the ubiquitin promoter.

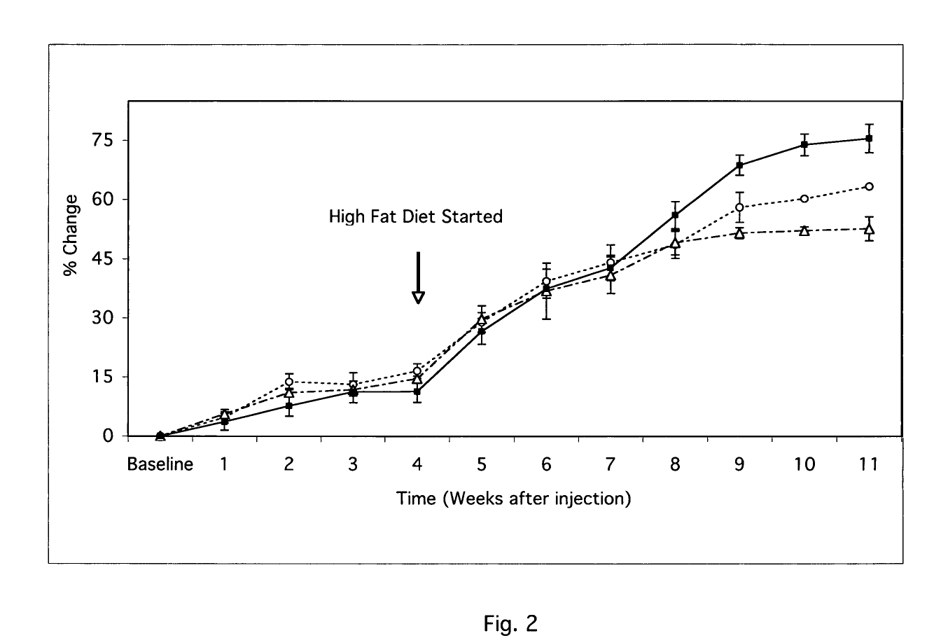

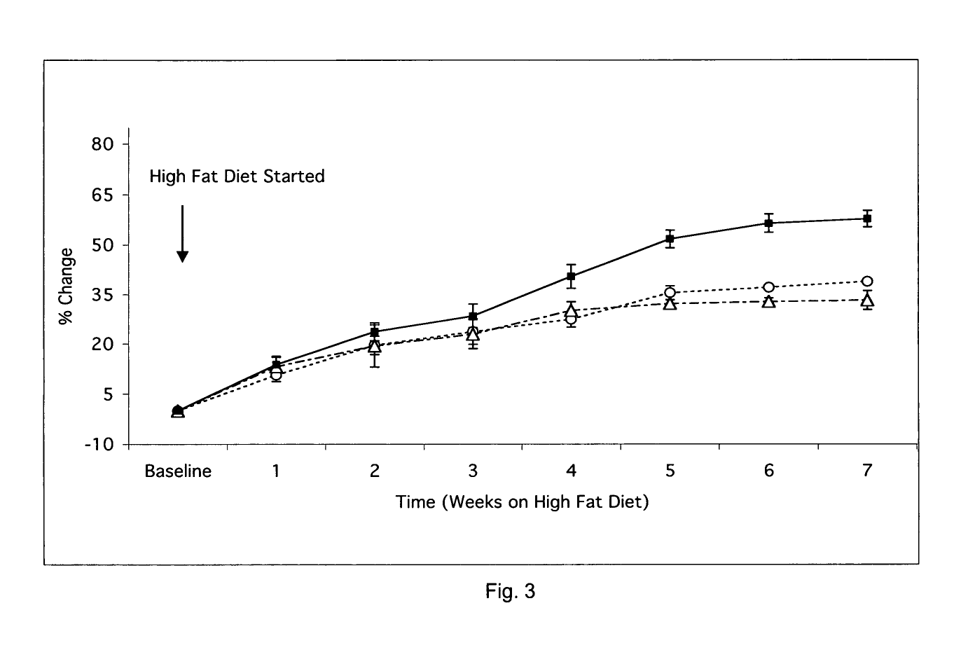

When fed a high fat diet, they normally rapidly gain 85-100% in body weight over a 12 week period and display elevated serum lipids and glucose profile reflective of an obese condition. Sham injected mice in these experiments also exhibited rapid bodyweight gain (FIGS. 2 and 3) as well as elevated serum glucose and lipid levels (Table 1). Injection of mice with hTSH-mFC-hα1 (SEQ ID NO:20) or hOGH-CTP-hα2 (SEQ ID NO:24) significantly attenuated this bodyweight gain on a high fat diet after 6-7 weeks and significantly attenuated serum glucose, cholesterol and insulin levels (Table 1). These changes correlate with the low, but significant, elevation of total T4 levels in the serum (Table 1). All samples were taken between the hours of 10-11 am after an 18 hr fast from mice of the same experiment. Data reported are the Mean+SEM (n=6) and * denoted statistical significance from sham control by Students t-test at p>0.05.

TABLE 1
Fasting Serum Chemistry Parameters of OGH/TSH-Fc DNA Tail Vein Injection mice.

| | Sham | mFc | hOGH—CTP-hα2 | [symbol] |
|---|---|---|---|---|
| Glucose (mg/dL) | 228.0 ± 16.3 | 184 ± 8.5 | 142.2 ± 24.1* | 115.4 ± 15.1* |
| Triglycerides (ng/ml) | 105.2 ± 4.3 | 101.6 ± 5.2 | 97.4 ± 14.1 | 94.8 ± 6.0 |
| Cholesterol (ng/ml) | 129.6 ± 5.7 | 126.4 ± 9.0 | 66.2 ± 11.3* | 66.9 ± 6.3* |
| NEFA (mEqu) | 0.7 ± 0.1 | 0.92 ± 0.04 | 0.9 ± 0.1 | 1.0 ± 0.1 |
| dLDL | 4.4 ± 0.6 | 5.3 ± 1.1 | 3.4 ± 0.4 | 3.6 ± 0.3 |
| Insulin (ng/ml) | 0.92 ± 0.26 | 1.41 ± 0.36 | 0.84 ± 0.36 | 0.67 ± 0.19 |
| T4 (ug/dL) | 3.7 ± 0.2 | 3.4 ± 0.3 | 7.2 ± 1.5* | 9.6 ± 0.9* |

1. An recombinant nucleic acid molecule encoding a thyroid-stimulating fusion polypeptide comprising R1, R2, wherein R1 is an orphan glycoprotein hormone (OGH), or an OGH family member, or a biologically active fragment thereof, R2 is a glycoprotein hormone subunit, or a biologically active fragment thereof, and further optionally comprising a peptide P) and/or a fusion component (F).

2. The nucleic acid of claim 1, wherein R1 is selected from the group consisting of human OGH (hOGH), human follicle stimulating hormone (hFSH), human thyroid stimulating hormone (hTSH), and human chorionic gonadotropin (hCG).

3. The nucleic acid of claim 1, wherein R2 is selected from the group consisting of α2, α1, and a fragment thereof capable of interacting with R1.

4. The nucleic acid of claim 1, wherein the optional fusion component (F) is selected from the group consisting of a multimerizing component, a serum protein, or a molecule capable of binding a serum protein.

5. The nucleic acid of claim 4, wherein the multimerizing component is selected from the group consisting of (i) an immunoglobulin-derived domain, (ii) a cleavable region (C-region), (ii) an amino acid sequence between 1 to about 500 amino acids in length, optionally comprising at least one cysteine residue, (iii) a leucine zipper, (iv) a helix loop motif, and (v) a coil-coil motif.

6. The nucleic acid of claim 5, wherein the immunoglobulin-derived domain is selected from the group consisting of the Fc domain of IgG and the heavy chain of IgG.

7. The nucleic acid of claim 6, wherein the Fc domain of IgG is human FcΔ1 (a).

8. The nucleic acid of claim 1, wherein P is a peptide between 1-50 amino acids in length.

9. The nucleic acid of claim 8, wherein P is a peptide between 3040 amino acids in length.

10. The nucleic acid of claim 9, wherein P comprises the C-terminal peptide of human chorionic gonadotropin.

11. The nucleic acid of claim 10, wherein P is SEQ ID NO:9.

12. The nucleic acid of claim 1, further comprising a sequence encoding a signal sequence (SS) component.

13. The nucleic acid of claim 1, wherein the components are arranged as R1-F-R2, R2-F-R1, R1-P-R2, R2-P-R1, R1-F-P-R2, R2-P-F-R1, R2-F-P-R1, R1-P-F-R2.

14. A vector comprising the nucleic acid molecule of claim 1.

15. A method of producing a fusion polypeptide, comprising culturing a host cell transfected with the vector of claim 14, under conditions suitable for expression of the protein from the host cell, and recovering the polypeptide so produced.

16. The method of claim 15, wherein the host cell is selected from the group consisting of a bacterial, yeast, insect, or mammalian cell.

17. The method of claim 16, wherein the host cell is selected from the group consisting of E. coli, B. subtilis, BHK, COS and CHO cells.

18. A fusion polypeptide encoded by the nucleic acid molecule of claim 1.

19. A fusion polypeptide comprising R1, R2, and P, wherein R1 is an orphan glycoprotein hormone (OGH), or an OGH family member, or a biologically active fragment thereof, R2 is a glycoprotein hormone subunit, or a biologically active fragment thereof, and P is a peptide; further optionally a fusion component (F).

20. The fusion polypeptide of claim 19, comprising an amino acid sequence selected from the group consisting of SEQ ID NOs:10 and 12.

21. A multimeric polypeptide comprising two or more of the fusion polypeptides of claim 19.

22. A pharmaceutical composition comprising the fusion polypeptide of claim 21 and a pharmaceutically acceptable carrier.

23. A therapeutic method for the treatment of an OGH-related disease or condition, comprising administering the pharmaceutical composition of claim 22 to a subject suffering from an OGH-related disease or condition.

24. The therapeutic method of claim 23, wherein the OGH-related disease or condition is selected from the group consisting of low metabolic rate, high serum triglycerides, high cholesterol, hypothyroidism, and obesity.

25. The therapeutic method for the treatment of the diseases or condition of claim 24 to moderately elevate T3/T4 levels to increase metabolic rate, confer resistance to a high fat diet and reduce body weight, elevated serum cholesterol, and/or triglycerides levels.


SEQUENCE LISTING (26 sequences)

SEQ ID NO: 1 - DNA, 414 bp, homo sapiens
atgactgctctctttctgatgtccatgctttttggccttgcatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtc

SEQ ID NO: 2 - protein, 138 aa, homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 3 - DNA, 708 bp, homo sapiens
ggagggggcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 4 - protein, 236 aa, homo sapiens
GGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - DNA, 278 bp, homo sapiens
dtcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 6 - protein, 92 aa, homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 7 - DNA, 390 bp, homo sapiens
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatc

SEQ ID NO: 8 - protein, 130 aa, homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 9 - DNA, 90 bp, homo sapiens
caggactcctcttcctcaaaggcccctccccccagccttccaagtccatcccgactcccggggccctcggacaccccgatcctcccacaa

SEQ ID NO: 10 - protein, 30 aa, homo sapiens
QDSSSSKAPPPSLPSPSRLPGPSDTPILPQ

SEQ ID NO: 11 - DNA, 321 bp, homo sapiens
caggaggcagtcatcccaggctgccacttacaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 12 - protein, 106 aa, homo sapiens
QEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 13 - DNA, 681 bp, homo sapiens
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 227 aa, homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1530 bp, Homo sapien
atgggagggggcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatccggcggaaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcaccggtcaggactcctcttcctcaaaggcccctccccccagccttccaagtccatcccgactcccggggccctcggacaccccgatcctcccacaaaccggtcaggaggcagtcatcccaggctgccacttacaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 16 - protein, 509 aa, Homo sapien
MGGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKSGGKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETITGQDSSSSKAPPPSLPSPSRLPGPSDTPILPQTGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 17 - DNA, 1596 bp, Homo sapien
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcaccggacaggactcctcttcctcaaaggcccctccccccagccttccaagtccatcccgactcccggggccctcggacaccccgatcctcccacaatccggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaaccggtcaggactcctcttcctcaaaggcccctccccccagccttccaagtccatcccgactcccggggccctcggacaccccgatcctcccacaaaccggtcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 18 - protein, 531 aa, Homo sapien [sequence omitted]

SEQ ID NO: 19 - DNA, 1413 bp, Homo sapien
atgactgctctctttctgatgtccatgctttttggccttgcatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtcaccggtggagggggcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaaccggtgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 20 - protein, 470 aa, Homo sapien
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSVTGGGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKTGAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 21 - DNA, 1431 bp, Homo sapien
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcaccggtggagggggcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaaccggtcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 22 - protein, 476 aa, Homo sapien
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETITGGGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKTGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 23 - DNA, 813 bp, Homo sapien
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcaccggtcaggactcctcttcctcaaaggcccctccccccagccttccaagtccatcccgactcccggggccctcggacaccccgatcctcccacaaaccggtcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 24 - protein, 270 aa, Homo sapien [sequence omitted]

SEQ ID NO: 25 - DNA, 717 bp, Homo sapien
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcaccggtcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactag

SEQ ID NO: 26 - protein, 238 aa, Homo sapien
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETITGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY